(19) United States
(12) Patent Application Publication
(10) Pub. No.: US 2026/0228897 A1
(43) Pub. Date: Aug. 6, 2026

(54) ITERATIVE SCHEME FOR ESTABLISHING OPTIMAL POSITION OF FOCUSING LENS AND RETINAL CAMERAS FOR IMPLEMENTING THE SAME

(71) Applicant: Verily Life Sciences LLC, Dallas, TX (US)

(72) Inventors: Alireza DASTGHEIB, Mountain View, CA (US); Karthik VIJAYRAGHAVAN, San Jose, CA (US)

(21) Appl. No.: 19/153,267

(22) PCT Filed: Feb. 28, 2024
(86) PCT No.: PCT/US2024/017600

§ 371 Date: Aug. 1, 2025

Related U.S. Application Data
(60) Provisional application No. 63/487,359, filed on Feb. 28, 2023.

(51) Int. Cl. G06T 7/00; A61B 3/12; A61B 3/14; G06T 5/70

(52) U.S. Cl. CPC G06T 7/0014; A61B 3/12; A61B 3/14; G06T 5/70; G06T 2207/10048; G06T 2207/30041; G06T 2207/30168

(57) ABSTRACT

Introduced here is an approach to autofocusing in which an iterative scheme for performing pairwise comparison of digital images is performed in order to identify the optimal position for the focusing lens. This approach relies on the fact that digital images generated in rapid succession are not fully independent, but are of the same subject with different degrees of blur or defocus. In accordance with this approach, a retinal camera can proceed through a series of steps, analyzing pairs of the digital images, until the optimal position for the focusing lens is found.

### CROSS-REFERENCE TO RELATED APPLICATIONS

This application claims priority to U.S. Provisional Application No. 63/487,359, titled “Iterative Scheme for Establishing Optimal Position of Focusing Lens and Retinal Camera for Implementing the Same” and filed on Feb. 28, 2023, which is incorporated by reference herein in its entirety.

### TECHNICAL FIELD

Various embodiments concern approaches to establishing the optimal position of a focusing lens of a retinal camera and computer programs for accomplishing the same.

### BACKGROUND

Fundus photography involves capturing a digital image of the fundus to document the retina, which is the neurosensory tissue in the eye that translates optical images into the electrical impulses that can be understood by the brain. The fundus can include the retina, optic disc, macula, fovea, and posterior pole.

Fundus cameras (also referred to as “retinal cameras”) are designed to provide an upright, magnified view of the fundus. FIG. 1 includes a high-level illustration of a retinal camera in operation. Generally, a subject (also referred to as a “patient” or “user”) will sit at the retinal camera with her chin set within a chin rest and her forehead pressed against a bar. An operator may be responsible for visually aligning the retinal camera and then pressing a shutter release that causes a digital image of the retina to be generated.

As shown in FIG. 1, light may be focused via a series of lenses through a masked aperture to form an annulus that passes through an objective lens onto the retina. The illuminating light rays are generated by one or more light sources, each of which is electrically coupled to a power source. When the objective lens is aligned with the retina, light reflected by the retina will pass through the un-illuminated hole in the annulus formed by the masked aperture. Normally, alignment is facilitated by having the patient place an eye proximate to a first eyepiece (also referred to as the “subject eyepiece,” “patient eyepiece,” or “user eyepiece”). As can be seen in FIG. 1, the optics of the retinal camera cause the illuminating light rays entering the eye and the imaging light rays exiting the eye to follow dissimilar paths.

The imaging light rays exiting the eye may initially be guided toward a second eyepiece (also referred to as the “operator eyepiece”) that is used by the operator to assist in aligning and/or focusing the illuminating light rays. When the operator presses the shutter release, a first mirror can interrupt the path of the illuminating light rays and a second mirror can fall in front of the operator eyepiece, which causes the imaging light rays to be redirected onto a capturing medium. Examples of capturing mediums include film, digital charge-coupled devices (“CCDs”), and complementary metal-oxide-semiconductors (“CMOSs”).

Healthcare professionals, such as optometrists, ophthalmologists, and orthoptists, may use the digital images generated by a retinal camera to detect or monitor diseases. For instance, these digital images may be used to document indicators of diabetes, glaucoma, and the like. Accordingly, it is critical that the digital images be high quality, so that these healthcare professionals can readily identify pathological features that are indicative of disease.

### BRIEF DESCRIPTION OF THE DRAWINGS

This patent or application contains at least one drawing executed in color. Copies of this patent or application publication with color drawings will be provided by the Office upon request and payment of the necessary fee.

FIG. 1 includes a high-level illustration of a retinal camera in operation.

FIG. 2 illustrates how a focusing lens can be repositioned by a motor along an axis that is roughly orthogonal to the capturing medium.

FIG. 3A depicts a scenario in which the lens tube of an optical assembly is aligned with the left eye.

FIG. 3B illustrates how the lens tube may retract through an aperture in an occlusion plate while moving between the left and right eyes.

FIG. 3C depicts a scenario in which the lens tube of the optical assembly is aligned with the right eye.

FIG. 4 illustrates how either a focusing lens may be repositioned by a focusing motor along an axis with respect to a capturing medium or a focusing assembly may be repositioned by the focusing motor along the axis with respect to the focusing lens.

FIG. 5 illustrates a network environment that includes a control platform that is executed by a retinal camera.

FIG. 6 illustrates an example of a retinal camera that is able to implement a control platform designed to, among other things, perform a focusing operation to establish an optimal location for a focusing lens.

FIG. 7 illustrates how the sharpness of a pair of digital images can be compared.

FIG. 8 shows how the amount of blur compares against the mean absolute difference between pairs of digital images in three different situations.

FIG. 9 includes a flow diagram of a process for identifying the optimal position for a focusing lens for imaging the eye of a subject.

FIG. 10 includes another flow diagram of a process for identifying the optimal position for a focusing lens for imaging the eye of a subject.

FIG. 11 includes a block diagram illustrating an example of a processing system that is able to implement at least some operations described herein.

Various features of the technology will become more apparent to those skilled in the art from a study of the Detailed Description in conjunction with the drawings. In the drawings, embodiments are illustrated by way of example and not limitation for the purpose of illustration. Those skilled in the art will recognize that alternative embodiments may be employed without departing from the principles of the present disclosure. Accordingly, while specific embodiments are shown in the drawings, the technology is amenable to various modifications.

### DETAILED DESCRIPTION

Imaging has historically been an effective means for detecting a variety of ailments. For instance, the digital images generated by retinal cameras are useful for detecting diseases, such as diabetes and glaucoma, because related complications, such as diabetic retinopathy and optic nerve damage, can be more readily discovered. Accurate diagnoses require that the digital images be sufficiently clear, and therefore quality of the digital images is paramount.

Historically, operators of retinal cameras were responsible for focusing the imaging light rays returning from the eyes of patients—generally by turning a knob in order to reposition a focusing lens responsible for directing or “focusing” the imaging light rays onto a capturing medium. This process was prone to errors. Some retinal cameras include a focusing motor (or simply “motor”) that repositions the focusing lens nearer to, or further from, the capturing medium in an automated manner. The processing by which the focusing lens is repositioned is commonly called “autofocusing.”

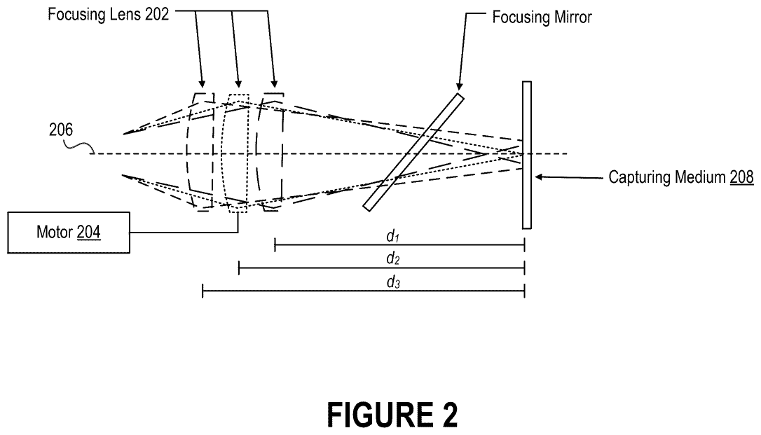

FIG. 2 illustrates how a focusing lens 202 can be repositioned by a motor 204 along an axis 206 that is roughly orthogonal to the capturing medium 208. If the focusing lens 202 were to be focused manually, the operator would look through the operator eyepiece and verify, with her eyes, whether the content appears sufficiently sharp. With autofocusing, focus is calculated electronically as the focusing lens 202 moves closer to, and further from, the capturing medium 208. Generally, digital images are continuously captured as the motor 204 sweeps the focusing lens 202 across a range of distances, as measured with respect to the capturing medium 208. A score that is representative of focus can then be calculated for each of the digital images, and the optimal position for the focusing lens 202 can be established by identifying which digital image had the highest score. Note that the term “highest score,” as used herein, is meant to refer to the score that is quantitatively determined to correspond to the highest focus, rather than any particular numeric direction.

Autofocusing represents a significant improvement over conventional approaches that rely heavily on operators for manually adjusting the focusing lens and reviewing the digital images. However, autofocusing tends to be a computationally intense process, as the motor 204 will sweep the focusing lens 202 across the entire range of distances and scores will be calculated for all of the digital images. In this way, each digital image is compared-via the scores with every other digital image. Consider, for example, the last digital image that is generated by the capturing medium 208. The score calculated for the last digital image must be compared against the scores calculated for all of the preceding digital images in order to determine whether the last digital image has the highest score.

Introduced here is an alternative approach to autofocusing in which an iterative scheme for performing pairwise comparison of digital images is performed in order to identify the optimal position for the focusing lens. This approach relies on the fact that digital images generated in rapid succession (e.g., over the course of several hundred milliseconds) are not fully independent, but are of the same subject with different degrees of blur or defocus. Because the digital images are generated in rapid succession, any unintentional movement (e.g., of the eye, head, or entire body) will be negligible in its impact on blurriness. Accordingly, digital images may be captured at a relatively higher frequency (e.g., 30, 60, or 120 digital images per second) for the purpose of establishing the optimal position for the focusing lens, while digital images may be captured at a relatively lower frequency (e.g., 5, 7, or 10 digital images per second) for the purpose of imaging an eye.

In accordance with this approach, a retinal camera (and more specifically, a computer program executing on the retinal camera) can proceed through a series of steps until the sharpest digital image is found. At each step, a pair of digital images can be compared against one another, and the retinal camera can identify the sharper digital image and then select the next pair of digital images accordingly. Generally, the retinal camera compares the sharper digital image against another digital image in a similar manner, and this process can continue until the sharpest digital image is found. Accordingly, the sharpest digital image may be identified after performing one-to-one comparisons in an iterative manner, a much less computationally intensive process than conventional approaches to autofocusing.

As an example, consider a scenario in which a pair of digital images—namely, a first digital image and a second digital image—of an eye of a subject are captured by a retinal camera under the same lighting condition. This lighting condition may be predetermined, or this lighting condition may be dynamically determined upon the individual presenting herself for imaging. Visible light or non-visible light (e.g., infrared light) may be emitted into the eye for imaging purposes, where the light reflected by the eye is used to create a digital image, whether for focusing or final imaging (e.g., for diagnostic analysis). However, it may be useful to use different lighting conditions for focusing and then final imaging. For example, infrared light may be shone into the eye and then the reflected infrared light may be used to generate digital images that are analyzed for the purpose of establishing an appropriate position for a focusing lens. If infrared light is used to illuminate the eye as part of the focusing operation, then the primary light source that provides visible illumination does not need to be moved relative to the eye—or even operated—during the focusing operation. After the focusing lens has been moved to the appropriate position, visible light may be shone into the eye and then the reflected visible light may be used to generate at least one digital image that is analyzed for the purpose of providing a diagnosis.

As part of a comparison operation, a computer program may produce a blurred version of the first digital image by providing the first digital image, as input, to a blurring algorithm. Similarly, the computer program may produce a blurred version of the second digital image by providing the second digital image, as input, to the blurring algorithm. For convenience, the blurred version of the first digital image may be referred to as the “first blurred digital image” while the blurred version of the second digital image may be referred to as the “second blurred digital image.” The computer program can then compare the first and second digital images against the first and second blurred digital images. Specifically, the computer program can compare the first blurred digital image against the second digital image, so as to generate a first metric that is indicative of the magnitude of difference between the first blurred digital image and second digital image. Moreover, the computer program can compare the second blurred digital image against the first digital image, so as to generate a second metric that is indicative of the difference between the second blurred digital image and first digital image. By comparing the first and second metrics, the computer program can establish whether the first digital image or second digital image is sharped. Said another way, the computer program can establish whether the first digital image or second digital image is sharper based on an analysis of the first and second metrics.

Assume, for example, that the first metric is either a singular value or plurality of values that quantitatively represent similarity between the first blurred digital image and second digital image, while the second metric is either a singular value or plurality of values that quantitatively represent similarity between the second blurred digital image and first digital image. If the magnitude of the second metric is larger than the magnitude of the first metric—indicating that there is a larger difference between the second blurred digital image and first digital image than between the first blurred digital image and second digital image—then the computer program may establish that the first digital image is sharper than the second digital image. Conversely, if the magnitude of the first metric is larger than the magnitude of the second metric—indicating that there is a larger difference between the first blurred digital image and second digital image than between the second blurred digital image and first digital image—then the computer program may establish that the second digital image is sharper than the first digital image.

As mentioned above, the retinal camera may generate a series of digital images as part of an autofocusing operation. By performing multiple iterations of the aforementioned comparison operation, the computer program can systematically identify the sharpest digital image and then take appropriate action. For example, the computer program may generate a signal that causes a motor to reposition the focusing lens in the position corresponding to the sharpest digital image.

Embodiments may be described with reference to particular types of imaging devices, imaging systems, network architectures, and the like. However, those skilled in the art will recognize that these features are similarly applicable to other types of imaging devices, imaging systems, and network architectures. For example, embodiments may be described in the context of a retinal camera that is responsible for imaging eyes for the purpose of detecting, diagnosing, or monitoring disease. However, the relevant features may be similarly applicable to digital single-lens reflex (“DSLR”) cameras that image items at different distances.

Moreover, while embodiments may be described in the context of computer-executable instructions, aspects of the technology could be implemented via hardware or firmware instead of, or in addition to, software. As an example, a computer program that resides on a retinal camera may be responsible for processing digital images generated by the retinal camera, determining sharpness of the digital images, and managing a motor that controls a focusing lens. The computer program may also be responsible for generating interfaces that guide or instruct an operator in operating the retinal camera. or the subject herself. Note that, in some embodiments, the retinal camera is designed to be operated by the subject whose eye is being imaged. Accordingly, the term “operator” could refer to the subject or another person, such as a healthcare professional or technician.

### Terminology

References in the present disclosure to “an embodiment” or “some embodiments” means that the feature, function, structure, or characteristic being described is included in at least one embodiment. Occurrences of such phrases do not necessarily refer to the same embodiment, nor do they necessarily refer to alternative embodiments that are mutually exclusive of one another.

The terms “comprise” and “comprising” are to be construed in an inclusive sense rather than an exclusive or exhaustive sense (i.e., in the sense of “including but not limited to”).

The term “based on” is also to be construed in an inclusive sense rather than an exclusive or exhaustive sense. Thus, unless otherwise noted, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively connected to one another despite not sharing a physical connection.

The term “module” may refer broadly to software, firmware, hardware, or combinations thereof. Modules are typically functional components that generate one or more outputs based on one or more inputs. A computer program may include or utilize one or more modules. For example, a computer program may utilize multiple modules that are responsible for completing different tasks, or a computer program may utilize a single module that is responsible for completing multiple tasks.

When used in reference to a list of items, the word “or” is intended to cover all of the following interpretations: any of the items in the list, all of the items in the list, and any combination of items in the list.

### Overview of Retinal Camera

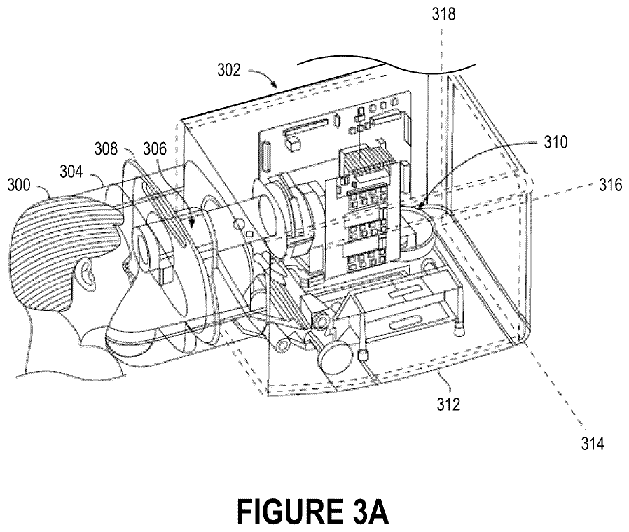

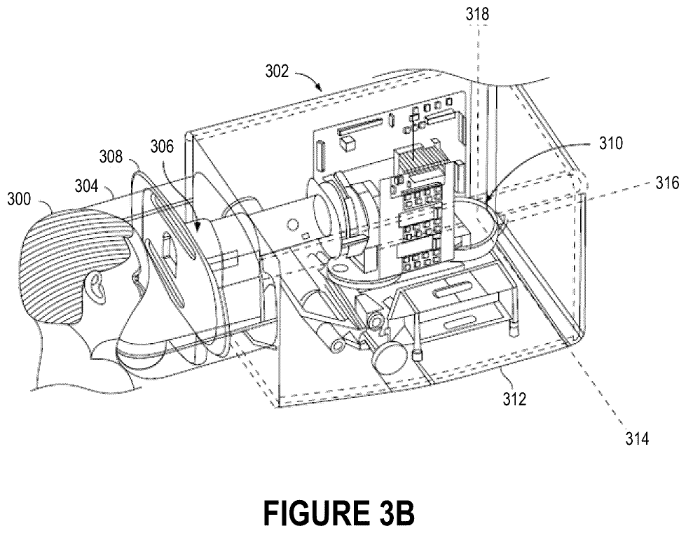

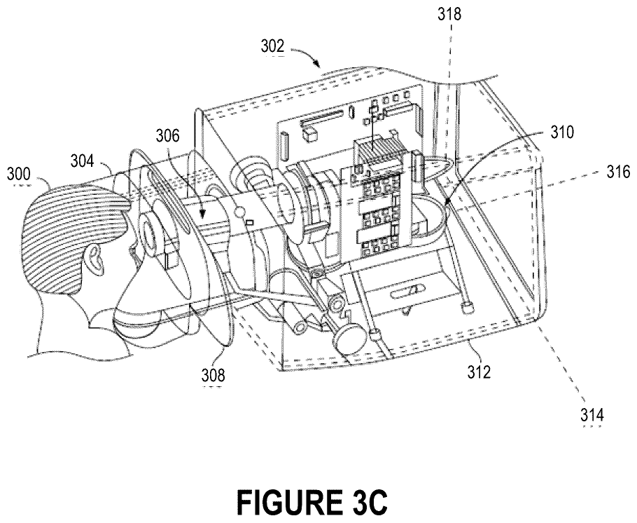

FIGS. 3A-C illustrate how an optical assembly of a retinal camera 302 can shift between multiple positions during an imaging operation so that both eyes can be imaged without requiring that an individual 300 move her head. In particular, after the individual 300 has placed her head against a flexible mask 304 designed to envelop the left and right eyes, the optical assembly of the retinal camera 302 can be moved, either manually or automatically, between multiple positions in order to generate images of the left and right eyes. FIG. 3A depicts a scenario in which the lens tube 306 of the optical assembly is aligned with the left eye, while FIG. 3C depicts a scenario in which the lens tube 306 of the optical assembly is aligned with the right eye. FIG. 3B illustrates how the lens tube 306 may retract through an aperture in an occlusion plate 308 while moving between the left and right eyes.

Several different technologies could be employed to facilitate the movement of the optical assembly between a first position corresponding to the left eye and a second position corresponding to the right eye.

Here, for example, the optical assembly is connected to a floating plate 310 that is attached to a rod. The rod is interconnected between the floating plate 310 and an interface component accessible along the bottom surface of the enclosure 312 of the retinal camera 302. An operator may be able to mechanically control the position of the optical assembly by manipulating the interface component. For example, the interface component may be movable between first and second positions corresponding to the first and second positions of the optical assembly.

In other embodiments, the retinal camera 302 includes one or more motors for moving the optical assembly or components of the optical assembly. For example, the retinal camera 302 may include a first controller responsible for controlling a first motor to cause movement along the x-axis 314, a second controller responsible for controlling a second motor to cause movement along the y-axis 316, or a third controller responsible for controlling a third motor to cause movement along the z-axis 318. As another example, the retinal camera 302 may include a multi-axis controller responsible for separately controlling multiple motors to cause movement along the x-, y-, and/or z-axes 314, 316, 318. The retinal camera 302 may include motors other than those responsible for causing movement along the x-, y-, and/or z-axes 314, 316, 318. For example, the retinal camera 302 may include a focusing motor that is responsible for causing movement of a focusing lens along the x-, y-, and/or z-axes 314, 316, 318 or a subset thereof (e.g., along the y-axis 316 only), as further discussed below. The focusing motor may be a stepper motor, a servo motor, or another type of electric motor.

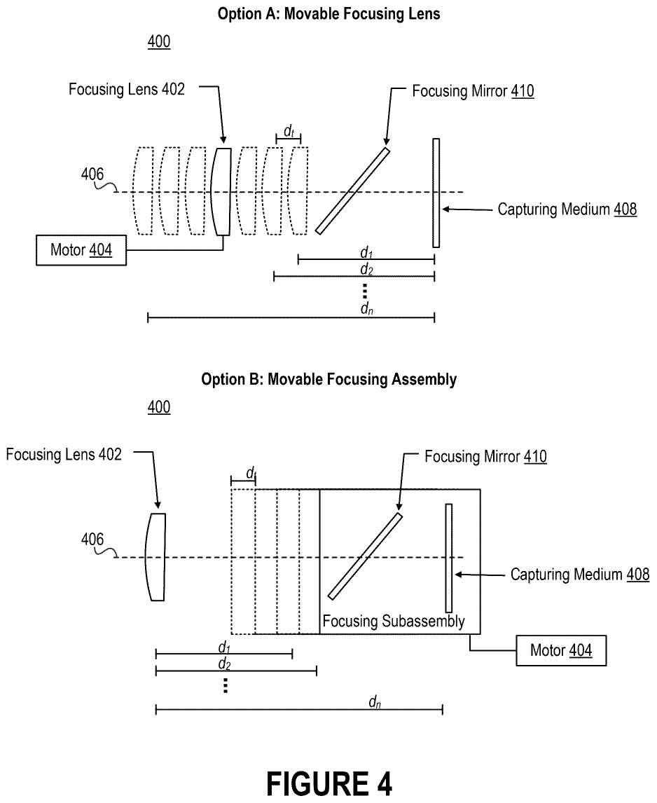

FIG. 4 illustrates how either a focusing lens 402 may be repositioned by a focusing motor 404 along an axis 406 with respect to a capturing medium 408 (Option A) or a focusing assembly—which includes the capturing medium 408—may be repositioned by the focusing motor 404 along the axis 406 with respect to the focusing lens 402 (Option B).

With Option A, the focusing lens 402 can be positioned in various locations, some of which are indicated using dotted lines. Each location may correspond to the focusing lens 402 being a different distance (e.g., d₁, d₂, . . . , dₙ) from the capturing medium 408. Accordingly, the focusing motor 404 may be able to move the focusing lens 402 closer to, and further from, the capturing medium 408 as necessary to achieve better sharpness.

The number of positions among which the focusing lens 402 can be repositioned may depend on the total range of movement. Assume, for example, that the leftmost and rightmost positions allow the focusing lens 402 to be positioned within a range of ±20 diopters. For the retinal camera, each diopter may correspond to displacement of 20 micrometers (“μm”), and therefore the full range of movement may be 800 μm. Note that each diopter could correspond to displacement of less than 20 μm or more than 20 μm in other embodiments. Thus, the full range of movement may be about 0.5-1 millimeter (“mm”). As an example, the leftmost or outermost position may cause the focusing lens 402 to be positioned 1 mm from the capturing medium 406 while the rightmost or innermost position causes the focusing lens 402 to be positioned 0.4 mm from the capturing medium 406, thereby resulting in a total range of 0.6 mm (i.e., 1 mm-0.4 mm) and a position spacing dt of 15 μm (i.e., 0.6 mm/40 diopters). The positions may be defined by a predetermined differential (e.g., every 15, 20, or 25 μm), or the positions may be defined by a predetermined count (e.g., 40, 50, or 60 different positions). Those skilled in the art will recognize that because movement of the focusing lens 402 is controlled by the focusing motor 404 in Option A, the number of positions and distance between positions may depend on the focusing motor 404. For example, some focusing motors may be able to reliably reposition the focusing lens 402 by distances as small as several μm while other focusing motors may not be able to achieve such resolution.

With Option B, the capturing medium 408 and focusing mirror 410 are part of a movable focusing subassembly. The focusing mirror 410 could be a beam splitter, for example, that allows some light to be transmitted therethrough (e.g., to the capturing medium 408) and reflects other light (e.g., along a path orthogonal to the axis 406). As shown in FIG. 4, the focusing motor 404 could move the focusing subassembly with respect to the focusing lens 402 that remains stationary, rather than moving the focusing lens 402 with respect to the capturing medium 408 and focusing mirror 410 as happened with Option A. The approach to repositioning may otherwise be similar, for example, in terms of full range of movement, position spacing, and the like.

Together, the focusing lens 402, focusing motor 404, capturing medium 408, and focusing mirror 410 may be called the “focusing assembly” of the retinal camera. In some embodiments, the focusing assembly—or at least a part thereof—is part of the optical assembly shown in FIGS. 3A-C, and therefore moves in concert with the optical assembly. In other embodiments, the focusing assembly is distinct from the optical assembly shown in FIGS. 3A-C, and therefore can be controlled independent of the optical assembly. In such embodiments, movement of the focusing assembly—or at least a part thereof—is independent of movement of the optical assembly, and vice versa.

### Overview of Control Platform

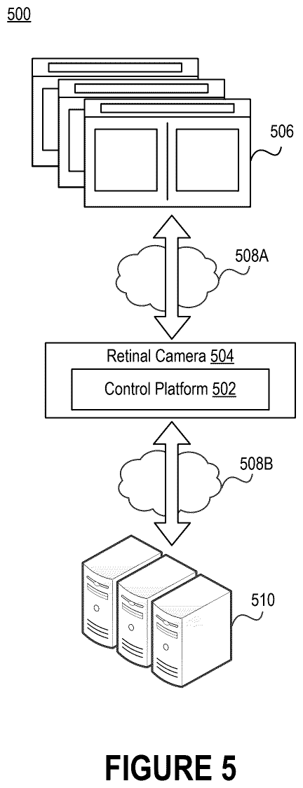

FIG. 5 illustrates a network environment 500 that includes a control platform 502 that is executed by a retinal camera 504. An individual (also called an “operator”) may be able to interact with the control platform 502 via interfaces 506. For example, an operator may be able to access an interface through which she can initiate a focusing operation in which the appropriate location of the focusing lens (e.g., focusing lens 402 of FIG. 4) is established. As another example, the operator may be able to access an interface through which she can review digital images generated by the retinal camera 504 or through which information regarding a subject can be presented. As mentioned above, the operator could be the subject herself in some scenarios.

As shown in FIG. 5, the control platform 502 may reside in a network environment 500. Thus, the retinal camera 504 on which the control platform 502 resides may be connected to one or more networks 508A-B. Depending on its nature, the retinal camera 504 could be connected to a personal area network (PAN), local area network (LAN), wide area network (WAN), metropolitan area network (MAN), or cellular network. For example, the retinal camera 504 could be connected to a server system 510 that is accessible via the Internet. Additionally or alternatively, the retinal camera 504 could be connected to a nearby computing device (e.g., associated with a patient, operator, or healthcare professional) in accordance with a short-range communication protocol, such as Bluetooth®, Near Field Communication (NFC), or the like.

While the interfaces 506 are generally presented on a display of the retinal camera 504, the interfaces 506 could alternatively or additionally be accessible via a web browser, desktop application, mobile application, or another form of computer program. For example, to interact with the control platform 502, an operator user may initiate a web browser on a computing device (e.g., a mobile phone or tablet computer), navigate to a web address associated with the control platform 502, and then view the interfaces 506. As another example, an operator user may access, via a desktop application, interfaces that are generated by the control platform 502 through which she can review digital images, review analyses of the digital images, and the like. Accordingly, interfaces generated by the control platform 502—or at least information acquired or produced by the control platform 502—may be viewable on various computing devices, including mobile phones, tablet computers, desktop computers, and the like.

Generally, the control platform 502 is representative of a computer program that is supported by a cloud computing service operated by, for example, Amazon Web Services®, Google Cloud Platform™, or Microsoft Azure®. Thus, the retinal camera 504 may be connected to a server system 510. Often, the server system 510 is comprised of multiple computer servers. These computer servers can include different types of data (e.g., information regarding patients, such as demographic information and health information), algorithms for processing, presenting, and analyzing digital images generated by retinal cameras, and other assets. Those skilled in the art will recognize that this data could also be distributed among the server system 510 and other computing devices, including the retinal camera 504. For example, digital images that are generated by the retinal camera 504 as part of a focusing operation may not be transmitted external to the retinal camera 504. As another example, digital images may be stored on, and initially processed by, the retinal camera 504, so that only high-quality digital images are transmitted external to the retinal camera 504 (e.g., to the server system 510) for further processing.

As mentioned above, aspects of the control platform 502 could be hosted locally, for example, in the form of a computer program executing on the retinal camera 504. Several different versions of computer programs may be available depending on the intended use. Assume, for example, that an operator would like to be actively guided through the process by which digital images of a patient are generated. In such a scenario, the computer program may allow for the review of digital images on the retinal camera 504. Such an approach may not only lessen the amount of data that needs to be transmitted external to the retinal camera 504, but can also allow the “guiding” to be performed in a more timely manner, as digital images need not be transmitted external to the retinal camera 504 for analysis. Alternatively, the digital images could be transmitted external to the retinal camera 504 (e.g., to the server system 510) for analysis and then additional actions, if any, could be taken based on input received by the retinal camera 504. For example, the focusing operation is normally performed by the retinal camera 504 in a semi- or fully-automated manner as further discussed below. However, there may be scenarios where assistance is provided by another computing device. For example, the retinal camera 504 may transmit the digital images generated as part of the focusing operation to the server system 510 for analysis, and the server system 510 may provide an indication of which digital image is sharpest or where the focusing lens should be optimally positioned. Such an approach may be beneficial in some scenarios (e.g., where the retinal camera 504 has few computational resources available) as “heavier” computations could be performed external to the retinal camera 504.

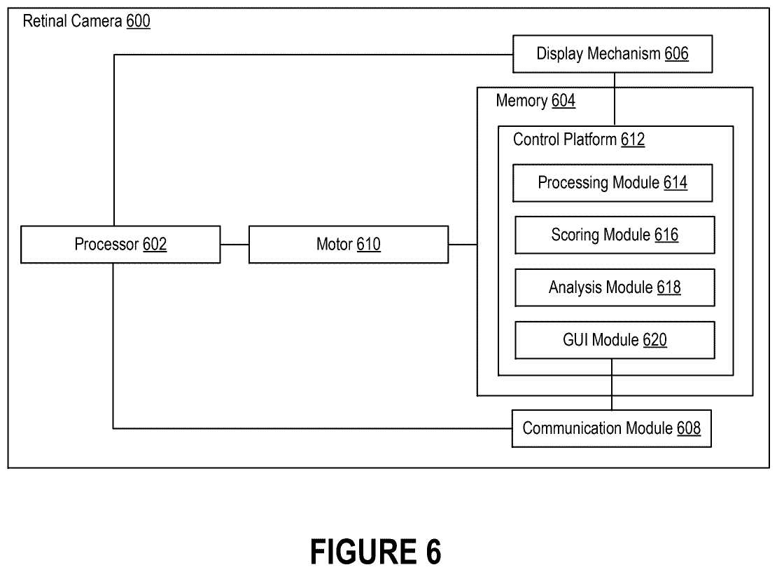

FIG. 6 illustrates an example of a retinal camera 600 that is able to implement a control platform 610 designed to, among other things, perform a focusing operation to establish an optimal location for a focusing lens (e.g., focusing lens 402 of FIG. 4). As shown in FIG. 6, the retinal camera 600 can include a processor 602, memory 604, display mechanism 606, communication module 608, and motor 610. Each of these components is discussed in greater detail below. For simplicity, various components of the retinal camera 600 are not shown in FIG. 6. For example, the light sources, lens, and capturing medium that allow for the generation of digital images are not shown in this simplified illustration of the retinal camera 600.

Those skilled in the art will recognize that different combinations of components may be present depending on the nature of the retinal camera 600. For example, some embodiments of the retinal camera 600 may include multiple display mechanisms, namely, an internal display mechanism that is observable by the patient while her eye is situated near the retinal camera 600 and an external display mechanism that is observable by the operator. However, if digital images—or analyses of the digital images—are to be viewed by the operator on another computing device rather than the retinal camera 600, then the retinal camera 600 may not include the external display mechanism.

The processor 602 can have generic characteristics similar to general-purpose processors, or the processor 202 may be an application-specific integrated circuit (“ASIC”) that provides control functions to the retinal camera 600. As shown in FIG. 6, the processor 602 can be coupled to all components of the retinal camera 600, either directly or indirectly, for communication purposes.

The memory 604 can be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 602, the memory 604 can also store data generated by the processor 602 (e.g., when executing the modules of the control platform 612). Note that the memory 604 is merely an abstract representation of a storage environment. The memory 604 could be comprised of actual integrated circuits (also called “chips”).

The display mechanism 606 can be any mechanism that is operable to visually convey information to an operator. For example, the display mechanism 606 can be a panel that includes light-emitting diodes (“LEDs”), organic LEDs, liquid crystal elements, or electrophoretic elements. As further discussed below, outputs produced by the control platform 612 (e.g., through execution of its modules) can be posted to the display mechanism 606 for review by a user of the retinal camera 600. In embodiments where the retinal camera 600 includes more than one display (e.g., an internal display and external display), the displays may not be identical to one another. For example, an external display may have larger dimensions or higher resolution than an internal display. As another example, the external display may be touch sensitive while the internal display may not be touch sensitive. Thus, the operator may be able to provide input to the control platform 610 by interacting with the external display, either directly or indirectly (e.g., via a control mechanism, such as a computer mouse, joystick, etc.). Meanwhile, the internal display is generally not interactable but is instead used to visually convey information to the patient over the course of an imaging operation.

The communication module 608 may be responsible for managing communications external to the retinal camera 600. The communication module 608 can be wireless communication circuitry that is able to establish wireless communication channels with other computing devices. Examples of wireless communication circuitry include 2.4 gigahertz (“GHz”) and 5 GHz chipsets compatible with Institute of Electrical and Electronics Engineers (“IEEE”) 802.11 also referred to as “Wi-Fi chipsets.” Alternatively, the communication module 608 may be representative of a chipset configured for Bluetooth, NFC, and the like.

The nature, number, and type of communication channels established by the retinal camera 600—and more specifically, the communication module 608—may depend on (i) the sources from which data is received by the control platform 612 and (ii) the destinations to which data is transmitted by the control platform 612. Assume, for example, that data generated by the control platform 612 is to be transmitted to a server and a mobile phone associated with the operator. In such embodiments, the communication module 608 may communicate with the server via the Internet using a Wi-Fi chipset and communicate with the mobile phone via a Bluetooth chipset.

For convenience, the control platform 612 is referred to as a computer program that resides in the memory 604. However, the control platform 612 could be comprised of software, firmware, or hardware that is implemented in, or accessible to, the retinal camera 600. In accordance with some embodiments described herein, the control platform 612 may include a processing module 614, a scoring module 616, an analysis module 618, and a graphical user interface (“GUI”) module 620. These modules could be integral parts of the control platform 612, or these modules could be logically separate from the control platform 612 but operate “alongside” it. Together, these modules enable the control platform 612 to perform a focusing operation with the goal of establishing the optimal location of a focusing lens.

The processing module 614 may be responsible for applying operations to the pixel data of digital images generated by the retinal camera 600. For example, the processing module 614 may process (e.g., denoise, filter, or otherwise alter) the pixel data so that it is usable by the other modules of the control platform 612. As mentioned above, the focusing operation may require that blurred versions of digital images generated by the retinal camera 600 be used to establish the sharpest digital image. The processing module 614 may be responsible for producing, deriving, or otherwise obtaining these blurred digital images. Assume, for example, that the retinal camera 600 generates a series of digital images as a focusing motor moves a focusing lens among a series of different positions. In such a scenario, the processing module 614 can produce a series of blurred digital images by providing the series of digital images, as input, to a blurring algorithm. The term “blurring algorithm,” as used herein, may be used to refer to an algorithm that when applied to a digital image, introduces blur to make the digital image less sharp. A blurring algorithm may rely on different approaches or functions to achieve blur, such as Gaussian blur (also called “Gaussian smoothing”), pixelation, defocusing, motion blurring, box blurring (also called “rectangular blurring”), and the like. As an example, a blurring algorithm that relies on a Gaussian blur may convolve a digital image that is provided as input with a Gaussian distribution, so as to produce a blurred digital image.

As further discussed below, a given digital image could be provided, as input, to the blurring algorithm multiple times in order to produce multiple blurred digital images based on the given digital image. For example, the processing module 614 may produce a first blurred digital image by providing the given digital image to the blurring algorithm as input, and the processing module 614 may produce a second blurred digital image by providing the first blurred digital image to the blurring algorithm as input. This process could be repeated to produce any number of blurred digital images for the given digital image.

The scoring module 616 may be responsible for performing pairwise comparisons of digital images generated by the retinal camera 600 as part of a focusing operation and corresponding blurred digital images produced by the processing module 614. Assume, for example, that the retinal camera 600 generates a pair of digital images—namely, a first digital image and a second digital image—as part of the focusing operation, and the processing module 614 produces a first blurred digital image for the first digital image and a second blurred digital image for the second digital image. The scoring module 616 can compare the first blurred digital image against the second digital image, so as to generate a first metric that is indicative of the magnitude of difference between the first blurred digital image and second digital image. Moreover, the scoring module 616 can compare the second blurred digital image against the first digital image, so as to generate a second metric that is indicative of the difference between the second blurred digital image and first digital image. Various algorithms or heuristics could be employed to compute the difference between two digital images. For example, the first metric may be representative of mean absolute difference between the first blurred digital image and second digital image, and the second metric may be representative of mean absolute difference between the second blurred digital image and first digital image.

The analysis module 618 may be responsible for analyzing the first and second metrics—also referred to as “scores”—in order to identify the sharper digital image. For example, the analysis module 618 may establish that the first digital image is sharper than the second digital image in response to a determination that the second metric is larger than the first metric. Conversely, if the analysis module 618 determines that the first metric is larger than the second metric, then the analysis module 618 may establish that the second digital image is sharper than the first digital image.

These steps can be iteratively performed by the scoring module 616 and analysis module 618, with the sharper digital image being compared against another digital image until all of the digital images generated as part of the focusing operation have been considered. Such an approach allows the sharpest digital image to be identified through iterative pairwise comparisons of the digital images generated as part of the focusing operation.

The GUI module 620 can generate interfaces that are viewable on the display mechanism 606. For example, the GUI module 620 could generate interfaces through which the operator can interact with the control platform 612, view outputs produced by the control platform 612, etc. Additionally, the GUI module 620 could generate interfaces through which information regarding the patient, the retinal camera 600, the imaging session, or individual digital images may be posted for presentation on the display mechanism 606.

Note that, in some embodiments, the control platform 612 is configured to produce, as output, raw digital images that are generated by the retinal camera 600. In other embodiments, the control platform 612 is configured to produce, as output, data objects that include pixel data corresponding to the digital images that are generated by the retinal camera 600. One example of a data object is a Digital Imaging and Communications in Medicine (“DICOM”) data object. In embodiments where the control platform 612 outputs DICOM data objects, each DICOM data object can include the pixel data corresponding to a digital image and context data related to attributes of the digital image. The processing module 614 may be responsible for populating the pixel data and context data into each DICOM data object. The context data may include information regarding the patient whose eye is captured in the digital image, the retinal camera 600 responsible for generating the digital image, the imaging session in which the digital image was generated, or the digital image itself.

### Methodologies for Iterative Autofocusing Scheme

At a high level, the scheme described herein relies on two primary concepts.

First, digital images generated as part of a focusing operation can be iteratively searched. This works in the context of autofocusing because it is reasonable to expect focus to be a roughly concave function with an optimum point. Therefore, it may be economical from a computational resource perspective to use a search optimization algorithm or numerical optimization algorithm rather than proceed through all of the digital images generated as part of the focusing operation. To accomplish this, a search optimization algorithm or numerical optimization algorithm may be applied by the control platform. One example of a numerical optimization algorithm is golden-section search.

Second, the digital images generated as part of the focusing operation can be compared in a pairwise manner at each iteration (also called a “step” of the focusing operation). The pairwise comparison could be accomplished by scoring each digital image individually and then comparing the scores against each other. Alternatively, the scoring step may be skipped altogether and the pair of digital images could be directly compared against one another as further discussed below. Pairwise comparison can also be an iterative process. When a first digital image is determined to be sharper than a second digital image, the first digital image can be compared against a third digital image (and so on, until some or all of the digital images generated as part of the focusing operation have been considered). When one digital image is much sharper than the other digital image, the comparison converges much more quickly, potentially saving time and computational resources.

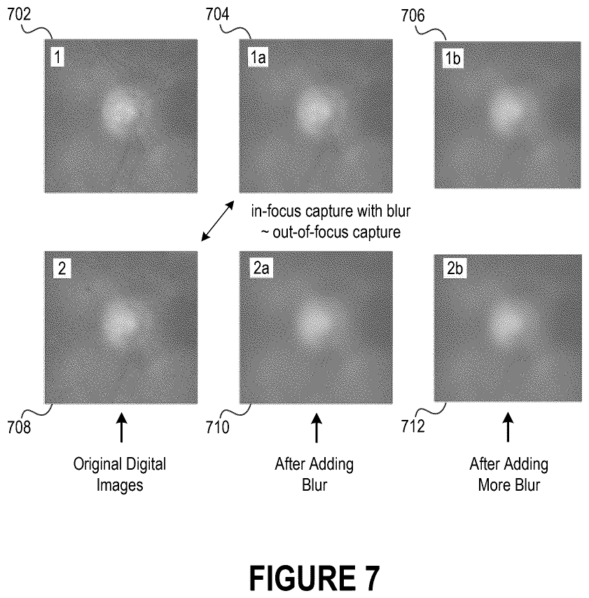

FIG. 7 includes several series of digital images. In the leftmost column, a pair of digital images—namely, a first digital image 702 and a second digital image 708—are shown. By progressively blurring the first digital image 702, the control platform can produce blurred digital images 704, 706. By progressively blurring the second digital image 708, the control platform can produce blurred digital images 710, 712.

To find the sharper of the first and second digital images 702, 708, the control platform can compare the absolute difference while progressively adding more blur to the first digital image 702, second digital image 708, or first and second digital images 702, 708. Thus, the control platform may compare the first digital image 702 against the second digital image 708, compare the first digital image 702 against blurred digital image 710, or compare the second digital image 708 against blurred digital image 704. With the added blur, the sharper digital image may come closer to the blurrier digital image than the blurrier image comes to the sharper digital image. FIG. 7 illustrates how the sharpness of a pair of digital images-namely, the first and second digital images 702, 708 can be compared. Note that this approach to pairwise comparison can be applied in other contexts.

As mentioned above, the first digital image 702 may be progressively blurred in order to produce blurred digital images 704, 706. Similarly, the second digital image 708 may be progressively blurred in order to produce blurred digital images 710, 712. To perform the pairwise comparison, the control platform may implement an algorithm that determines the difference between the second digital image 708 and blurred digital image 704, determines the difference between the first digital image 702 and blurred digital image 710, and compares these differences. In the example shown in FIG. 7, blurred digital image 704 is more similar to second digital image 708 than blurred digital image 710 is to first digital image 702. This means that blurring the first digital image 702 brings it closer to the second digital image 708, whereas blurring the second digital image 708 causes it to be further from the first digital image 702. Therefore, the control platform may determine that the first digital image 702 is sharper than the second digital image 708.

This process can continue until a desired level of distinction is achieved. Note that the amount of blur applied at each step can be adjusted based on, for example, the computational resources available to the control platform. Thus, the amount of blur can be “tuned.” Similarly, the number of steps—as measured in the number of times that the blurring algorithm is applied to a given digital image—can be adjusted based on, for example, how quickly the focusing operation is to be completed. Generally, fewer applications of the blurring algorithm will cause the focusing operation to be completed more quickly, though the differences between a given pair of digital images may be smaller with fewer applications of the blurring algorithm.

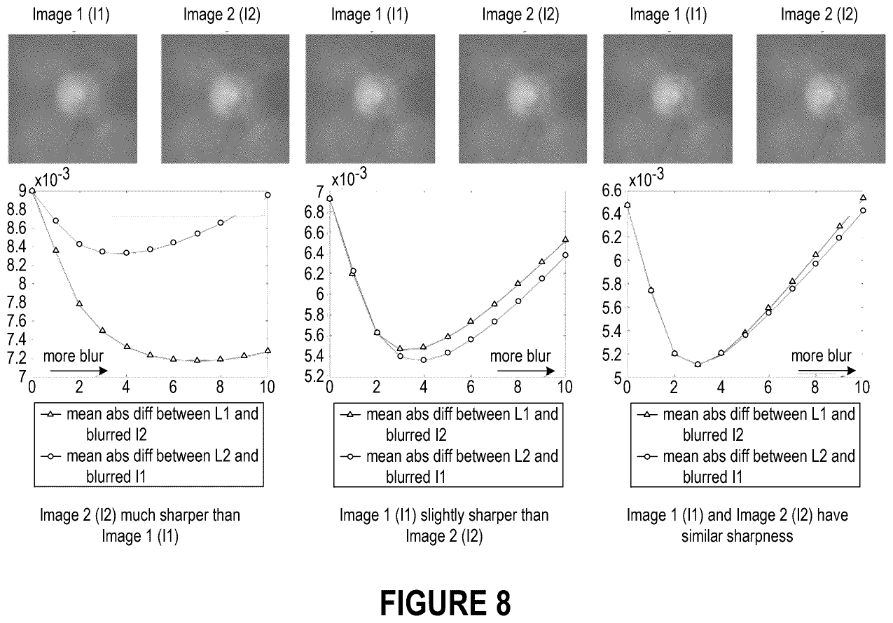

FIG. 8 shows how the amount of blur compares against the mean absolute difference between pairs of digital images in three different situations.

The leftmost chart corresponds to a scenario where the second digital image (i.e., I2) is much sharper than the first digital image (i.e., I1). As can be seen in FIG. 8, with no blur, the first and second digital images are close to one another, with a difference on the order of roughly 9.0×10⁻³. Adding more blur causes the mean absolute difference between the first digital image and blurred second digital image to diverge from the mean absolute difference between the second digital image and blurred first digital image. If the blurring algorithm is applied a single time, the difference is roughly 0.3×10⁻³ (i.e., 8.7×10⁻³-8.4×10⁻³). However, if the blurring algorithm is applied four times, the difference is roughly 1.0×10⁻³ (i.e., 8.4×10⁻³-7.4×10⁻³).

The middle chart corresponds to a scenario where the first digital image is sharper than the second digital image, albeit by a smaller magnitude than the leftmost chart. As can be seen in FIG. 8, there is little difference in the mean absolute difference between the first digital image and blurred second digital image and the mean absolute difference between the second digital image and blurred first digital image until the blurring algorithm is applied three times. In a scenario where the mean absolute differences are within a threshold amount (e.g., 0.1×10⁻³ or 0.25×10⁻³), the control platform may reapply the blending algorithm. This can be done in each step, as necessary. Accordingly, the blending algorithm may be applied once in some steps, and the blending algorithm may be applied multiple times in other steps.

The rightmost chart corresponds to a scenario where the first and second digital images have similar sharpness. As can be seen in FIG. 8, there is little separation in the mean absolute difference between the first digital image and blurred second digital image and the mean absolute difference between the second digital image and blurred first digital image, even after the blending algorithm has been applied five times. There is some separation after the blending algorithm is applied six times; however, the fact that the first and second digital images are still comparable until that amount of blending is applied indicates that the first and second digital images are very similar.

If the control platform had no constraints in terms of time or computational resources, then the blending algorithm could be repeatedly applied until there is meaningful separation between the mean absolute difference between the first digital image and blurred second digital image and the mean absolute difference between the second digital image and blurred first digital image. However, that is rarely the case. Accordingly, the control platform may be programmed such that the blending algorithm is applied no more than a predetermined number (e.g., 2, 3, 5) of times. If the mean absolute difference between the first digital image and blurred second digital image is still comparable to the mean absolute difference between the second digital image and blurred first digital image after the blending algorithm has been applied the predetermined number of times, then the control platform may implement a heuristic or rule for identifying the clearer digital image. For example, the control platform may identify whichever digital image was the “winning” digital image in the previous step as the clearer digital image. As another example, the control platform may identify whichever digital image is associated with the larger mean absolute difference as the clearer digital image. Accordingly, applying the blurring algorithm multiple times generally makes it easier to determine which of the first and second digital images is sharper. However, this must be weighed against the additional time needed to apply the blurring algorithm multiple times, as well as the additional computational resources needed to apply the blurring algorithm multiple times.

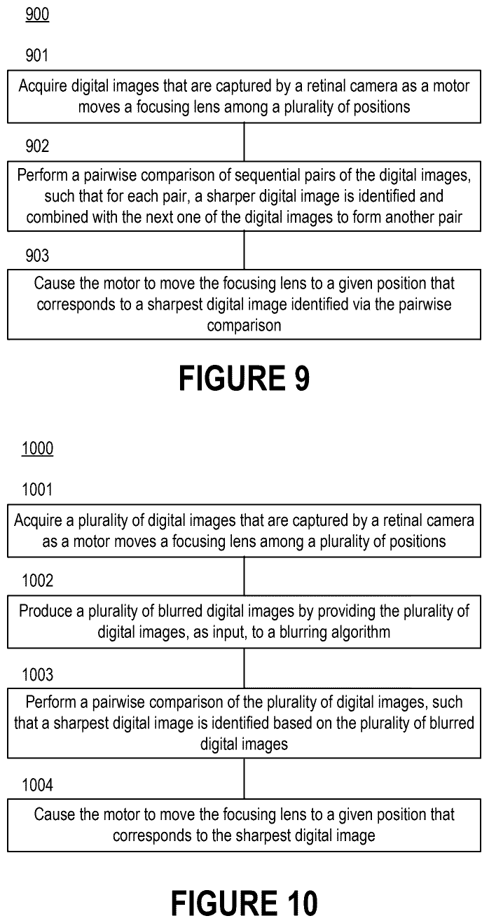

FIG. 9 includes a flow diagram of a process 900 for identifying the optimal position for a focusing lens for imaging the eye of a subject. Initially, a control platform can acquire digital images that are captured by a retinal camera as a motor moves the focusing lens among a plurality of positions (step 901). Each of the digital images may be associated with a different one of the plurality of positions. As mentioned above, the control platform is generally implemented on, and executed by, the retinal camera, though the control platform could be external to the retinal camera.

The control platform can then perform a pairwise comparison of sequential pairs of the digital images, such that for each pair, the sharper digital image is identified and combined with the next one of the digital images to form another pair (step 902) while the less-sharp digital image is discarded. Assume, for example, that the digital images are arranged in sequential order, from a first digital image to a last digital image. The first digital image can be compared against the second digital image, and whichever of the first and second digital images is sharper can then be compared against the third digital image while whichever of the first and second digital images is less sharp can be discarded. This process can be performed iteratively, with one digital image being discarded in each iteration, until some or all of the digital images have been considered.

Note that the process 900 could be performed similarly if the pairs of digital images being compared are not in sequential order. Assume, for example, that the control platform acquires a set of digital images in sequential order. In such a scenario, the control platform could segment the digital images into multiple subsets, each of which includes a different subset of the set of digital images in sequential order. The process 900 could be performed for each of the multiple subsets, such that a sharpest digital image is identified for each of the multiple subsets. Then, the process 900 could be compared in a pairwise manner, for example, beginning with the sharpest digital images identified for the first two subsets. Alternatively, the control platform could compare digital images selected from across the set (e.g., every fifth, tenth, or twentieth digital image) in a pairwise manner in order to identify an interval that likely includes the sharpest digital image. Assume, for example, that every fifth digital image is selected for comparison purposes. If the tenth digital image is determined to be the sharpest, then the interval may be defined from the sixth digital image to the fourteenth digital image. Within the interval, the control platform could perform a pairwise comparison of sequential pairs of digital images.

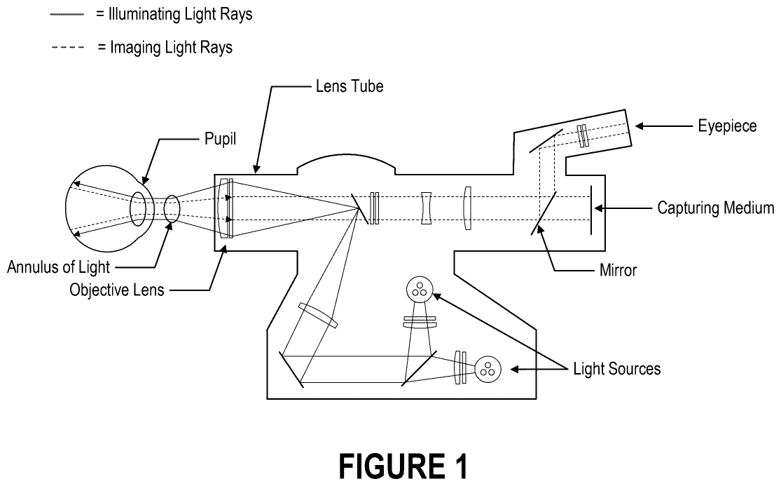

The benefits of this approach can be more readily understood through a comparison to the conventional approach to retinal imaging. In order for an eye to be imaged using a conventional retinal camera, a subject sits at the conventional retinal camera with her chin set within a chin rest and her forehead pressed against a bar, as discussed above with reference to FIG. 1. The subject will commonly be restrained in this position, while an operator visually aligns the objective lens with the eye and then presses a shutter release to capture a single digital image of high quality.

However, if the subject is largely or entirely responsible for orienting her own eye with respect to the objective lens, this approach is simply impractical. Because capturing a single digital image of high quality is difficult, a better approach involves taking a series of digital images in rapid succession (e.g., over the course of several hundred milliseconds) as part of a burst imaging operation and then identifying the highest quality digital image from among the series of digital images. Processing and storing the entire series of digital images can be “costly” in terms of available computational resources and memory space, and therefore the approach introduced here may be used to not only identify the highest quality digital image but also discard digital images having lower quality in an efficient, timely manner.

The nature of step 902 may vary depending on how similar the digital images being compared are to one another. For each sequential pair that includes a first digital image and a second digital image, the control platform can produce (i) a first blurred digital image by providing the first digital image, as input, to a blurring algorithm and (ii) a second blurred digital image by providing the second digital image, as input, to the blurring algorithm. Then, the control platform can compute (i) a first metric that is indicative of the magnitude of difference between the first blurred digital image and the second digital image and (ii) a second metric that is indicative of the magnitude of difference between the second blurred digital image and the first digital image.

If the first metric is not within a threshold amount of the second metric, then the control platform can identify the sharper digital image and proceed to the next iteration of step 902. The threshold amount could be a static value, or the threshold amount could be dynamically determined based on the first metric or the second metric. For example, the threshold amount may be representative of a predetermined percentage of the first metric or second metric.

However, if the first metric is within a threshold amount of the second metric, additional blur may be applied to the first and second digital images. Specifically, the control platform may produce a third blurred digital image by providing the first blurred digital image, as input, to the blurring algorithm, and the control platform may produce a fourth blurred digital image by providing the second blurred digital image, as input, to the blurring algorithm. The control platform can then compute (i) a third metric that is indicative of the magnitude of difference between the third blurred digital image and the second digital image and (ii) a fourth metric that is indicative of the magnitude of difference between the fourth blurred digital image and the first digital image. Again, the control platform can determine the difference between the third and fourth metrics. If the third metric is not within the threshold amount of the fourth metric, then the platform can identify the sharper digital image and proceed to the next iteration of step 902. However, if the third metric is within the threshold amount of the fourth metric, this process can be repeated again. As discussed above with reference to FIG. 8, some pairs of digital images may only require a single application of the blurring algorithm, while other pairs of digital images may require multiple applications of the blurring algorithm.

Note that, in some embodiments, the control platform performs pairwise comparing of sequential pairs of digital images until all of the digital images have been considered. In other embodiments, only some of the digital images are considered. For example, the control platform may perform pairwise comparing until a numerical optimization algorithm identifies a local maximum difference between the metrics generated for each sequential pair. This can work because it is generally reasonable to expect focus to be a roughly concave function with an optimum point, and therefore any local minimum can also be assumed to be a global minimum.

Thereafter, the control platform can cause the motor to move the focusing lens to a given position that corresponds to the sharpest digital image identified via the pairwise comparison (step 903). For example, the control platform may generate an instruction that prompts or provokes the motor to move the focusing lens to the given position, so that another digital image can be generated with the focusing lens located in the given position. This other digital image may be generated under the same lighting conditions as the digital images compared against one another or a different lighting condition. For example, the digital images to be compared against one another may be generated via illumination with non-visible light (e.g., infrared light), and then after the focusing lens is located in the given position, another digital image may be generated via illumination with visible light.

FIG. 10 includes another flow diagram of a process 1000 for identifying the optimal position for a focusing lens for imaging the eye of a subject. Initially, a control platform can acquire a plurality of digital images that are captured by a retinal camera as a motor moves a focusing lens among a plurality of positions (step 1001). Each digital image of the plurality of digital images may be associated with a different one of the plurality of positions.

The control platform can then produce a plurality of blurred digital images by providing the plurality of digital images, as input, to a blurring algorithm (step 1002). At a high level, the blurring algorithm smoothens the color transition across a given image. The blurring algorithm may utilize a box blur, a Gaussian blur, or another form of filter that implements a different type of blur. In some embodiments, the control platform produces the plurality of blurred digital images in response to acquiring the plurality of digital images. In other embodiments, the control platform produces blurred digital images only when necessary. For example, the control platform may produce blurred digital images only for the pair of digital images that are presently being compared. Such an approach may allow for further reduction in consumption of computational resources, especially if the control platform is programmed to employ a numerical optimization algorithm to establish when to conclude the pairwise comparison process.

Then, the control platform can perform a pairwise comparison of the plurality of digital images, such that a sharpest digital image is identified based on the plurality of blurred digital images (step 1003). Step 1003 of FIG. 10 may be comparable to step 902 of FIG. 9. As mentioned above, the plurality of digital images may be generated in sequential order, from a first digital image to a last digital image. Similarly, the plurality of blurred digital images may be produced in sequential order, from a first blurred digital image to a last blurred digital image. To perform the pairwise comparison, the control platform may implement a scheme in which (i) the first blurred digital image is compared against a second digital image that immediately follows the first digital image, to generate a first metric that is indicative of the magnitude of difference between the first blurred digital image and the second digital image, (ii) a second blurred digital image that immediately follows the first blurred digital image is compared against the first digital image, to generate a second metric that is indicative of the magnitude of difference between the second blurred digital image and the first digital image, and (iii) a sharper digital image is identified from among the first and second digital images based on an analysis of the first and second metrics. As discussed above, the scheme can be implemented iteratively, with the sharper digital image identified in each instance of the scheme being compared against the next digital image, until the sharpest digital image has been identified from among the plurality of digital images. Alternatively, the scheme can be implemented iteratively, with the sharper digital image identified in each instance of the scheme being compared against the next digital image, until the first and second metrics converge within a threshold amount. As another example, the scheme can be implemented iteratively, with the sharper digital image identified in each instance of the scheme being compared against the next digital image, until a predetermined amount of time elapses. Following the conclusion of the predetermined amount of time, whichever digital image is presently identified as sharpest may be identified as the sharpest digital image.

After the sharpest digital image has been identified, the control platform can cause the motor to move the focusing lens to a given position that corresponds to the sharpest digital image (step 1004). For example, the control platform may generate an instruction that prompts or provokes the motor to move the focusing lens to the given position, as mentioned above.

Unless contrary to physical possibility, it is envisioned that the steps described above may be performed in various sequences and combinations. For example, blurred digital images could be produced at the same time for all of the digital images captured as part of the focusing operation, or blurred digital images could be produced, as necessary, as part of each step of the pairwise comparison operation as discussed above. Accordingly, steps could be added to, or removed from, the processes described above. Other steps may also be included in some embodiments.

### Processing System

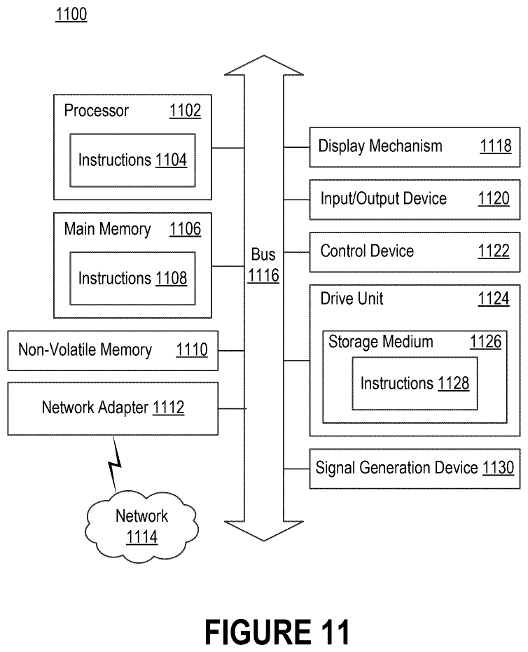

FIG. 11 includes a block diagram illustrating an example of a processing system 1100 that is able to implement at least some operations described herein. For example, components of the processing system 1100 may be included in a retinal camera or another computing device on which a control platform is stored and executed.

The processing system 1100 may include a processor 1102, main memory 1106, non-volatile memory 1110, network adapter 1112, display mechanism 1118, input/output device 1120, control device 1122 (e.g., a keyboard, pointing device, or mechanical input such as a button), drive unit 1124 that includes a storage medium 1126, or signal generation device 1130 that are communicatively connected to a bus 1116. The bus 1116 is illustrated as an abstraction that represents one or more physical buses and/or point-to-point connections that are connected by appropriate bridges, adapters, or controllers. The bus 1116, therefore, can include a system bus, Peripheral Component Interconnect (“PCI”) bus, PCI-Express bus, HyperTransport bus, Industry Standard Architecture (“ISA”) bus, Small Computer System Interface (“SCSI”) bus, Universal Serial Bus (“USB”), Inter-Integrated Circuit (“I²C”) bus, or a bus compliant with IEEE Standard 1394.

While the main memory 1106, non-volatile memory 1110, and storage medium 1126 are shown to be a single medium, the terms “storage medium” and “machine-readable medium” should be taken to include a single medium or multiple media that stores instructions 1104, 1108, 1128. The terms “storage medium” and “machine-readable medium” should also be taken to include any medium that is capable of storing, encoding, or carrying a set of instructions for execution by the processing system 1100.

In general, the routines executed to implement the embodiments of the present disclosure may be implemented as part of an operating system or a specific computer program. Computer programs typically comprise one or more instructions (e.g., instructions 1104, 1108, 1128) set at various times in various memories and storage devices in a computing device. When read and executed by the processor 1102, the instructions cause the processing system 1100 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile memory and non-volatile memory 1110, removable disks, hard disk drives, optical disks (e.g., Compact Disk Read-Only Memory (“CD-ROM”) and Digital Versatile Disks (“DVDs”)), cloud-based storage, and transmission-type media such as digital and analog communication links.

The network adapter 1112 enables the processing system 1100 to mediate data in a network 1114 with an entity that is external to the processing system 1100 through any communication protocol supported by the processing system 1100 and the external entity. The network adapter 1112 can include a network adaptor card, a wireless network interface card, a switch, a protocol converter, a gateway, a bridge, a hub, a receiver, a repeater, or a transceiver that includes a wireless chipset (e.g., enabling communication over Bluetooth or Wi-Fi).

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A method comprising:
acquiring a pair of digital images of an eye of a patient captured by a retinal camera under a same lighting condition, the pair of digital images including a first digital image and a second digital image;
producing a first blurred digital image by providing the first digital image, as input, to a blurring algorithm;
producing a second blurred digital image by providing the second digital image, as input, to the blurring algorithm;
generating a first metric that is indicative of a magnitude of difference between the first blurred digital image and the second digital image by comparing the first blurred digital image against the second digital image;
generating a second metric that is indicative of a magnitude of difference between the second blurred digital image and the first digital image by comparing the second blurred digital image against the first digital image;
establishing that the first digital image is sharper than the second digital image based on an analysis of the first and second metrics; and
discarding the second digital image.

2. The method of claim 1,
wherein the first metric is representative of mean absolute difference between the first blurred digital image and the second digital image, and
wherein the second metric is representative of mean absolute difference between the second blurred digital image and the first digital image.

3. The method of claim 1, wherein the pair of digital images are captured in succession, with the first digital image being captured before the second digital image.

4. The method of claim 1, wherein the same lighting condition involves illuminating the eye with infrared light, such that the first digital image is representative of infrared light reflected by the eye at a first point in time and the second digital image is representative of infrared light reflected by the eye at a second point in time.

5. The method of claim 4,
wherein the first digital image is captured while a focusing lens is located in a first position, and
wherein the second digital image is captured while the focusing lens is located in a second position different than the first position.

6. The method of claim 5, further comprising:
in response to said establishing,
causing a motor to reposition the focusing lens in the first position; and
causing a third digital image to be captured while visible light is shone into the eye, such that the third digital image is representative of visible light reflected by the eye at a third point in time.

7. A non-transitory medium with instructions stored thereon that, when executed by a processor of a retinal camera, cause the retinal camera to perform operations comprising:
acquiring digital images that are captured as a motor moves a focusing lens among a plurality of positions,
wherein each of the digital images is associated with a different one of the plurality of positions;
performing a pairwise comparison of pairs of the digital images, such that for each pair, a sharper digital image is identified and combined with a next one of the digital images to form another pair; and
causing the motor to move the focusing lens to a given position that corresponds to a sharpest digital image among the digital images that is identified via the pairwise comparison.

8. The non-transitory medium of claim 7, wherein the operations further comprise:
causing a digital image to be captured for diagnostic purposes in response to a determination that the focusing lens is located in the given position.

9. The non-transitory medium of claim 8,
wherein the digital images are generated by the retinal camera based on non-visible light that is shone into, and reflected by, an eye presented to the retinal camera, and
wherein the digital image is generated by the retinal camera based on visible light that is shone into, and reflected by, the eye presented to the retinal camera.

10. The non-transitory medium of claim 7, wherein said performing comprises:
for each pair that includes a first digital image and a second digital image,
producing a first blurred digital image by providing the first digital image, as input, to a blurring algorithm,
producing a second blurred digital image by providing the second digital image, as input, to the blurring algorithm,
comparing the first blurred digital image against the second digital image, so as to generate a first metric that is indicative of a magnitude of difference between the first blurred digital image and the second digital image,
comparing the second blurred digital image against the first digital image, so as to generate a second metric that is indicative of a magnitude of difference between the second blurred digital image and the first digital image, and
identifying the sharper digital image via analysis of the first and second metrics.

11. The non-transitory medium of claim 10, wherein said performing is performed until a numerical optimization algorithm identifies a local maximum difference between the first and second metrics generated for each pair.

12. The non-transitory medium of claim 7, wherein said performing comprises:
for each pair that includes a first digital image and a second digital image,
producing a first blurred digital image by providing the first digital image, as input, to a blurring algorithm,
producing a second blurred digital image by providing the second digital image, as input, to the blurring algorithm,
computing (i) a first metric that is indicative of a magnitude of difference between the first blurred digital image and the second digital image and (ii) a second metric that is indicative of a magnitude of difference between the second blurred digital image and the first digital image,
determining that the first metric is within a threshold amount of the second metric,
producing a third blurred digital image by providing the first blurred digital image, as input, to the blurring algorithm,
producing a fourth blurred digital image by providing the second blurred digital image, as input, to the blurring algorithm,
computing (i) a third metric that is indicative of a magnitude of difference between the third blurred digital image and the second digital image and (ii) a fourth metric that is indicative of a magnitude of difference between the fourth blurred digital image and the first digital image, and
identifying the sharper digital image via analysis of the third and fourth metrics.

13. The non-transitory medium of claim 12, wherein the threshold amount is a static value.

14. The non-transitory medium of claim 12, wherein the threshold amount is dynamically determined based on the first metric or the second metric.

15. A non-transitory medium with instructions stored thereon that, when executed by a processor, cause the processor to perform operations comprising:
acquiring a plurality of digital images that are captured by a retinal camera under a first lighting condition as a motor moves a focusing lens among a plurality of positions,
wherein each digital image of the plurality of digital images is associated with a different one of the plurality of positions;
producing a plurality of blurred digital images by providing the plurality of digital images, as input, to a blurring algorithm;
performing a pairwise comparison of the plurality of digital images, such that a sharpest digital image among the plurality of digital images that is identified based on an analysis of the plurality of blurred digital images; and
causing the motor to move the focusing lens to a given position that corresponds to the sharpest digital image; and
causing the retinal camera to capture a digital image, with the focusing lens in the given position, under a second lighting condition.

16. The non-transitory medium of claim 15,
wherein the plurality of digital images are captured in a first sequential order, from a first digital image to a last digital image, and
wherein the plurality of blurred digital images are produced in a second sequential order, wherein the first sequential order maps to the second sequential order.

17. The non-transitory medium of claim 16, wherein said performing comprises:
implementing an image comparison scheme in which-a
first blurred digital image is compared against a second digital image that immediately follows the first digital image in the first sequential order, to generate a first metric that is indicative of a magnitude of difference between the first blurred digital image and the second digital image,
a second blurred digital image that immediately follows the first blurred digital image in the second sequential order is compared against the first digital image, to generate a second metric that is indicative of a magnitude of difference between the second blurred digital image and the first digital image,
a sharper digital image is identified from among the first and second digital images based on an analysis of the first and second metrics.

18. The non-transitory medium of claim 17, wherein said implementing is performed iteratively, with the sharper digital image identified in each instance of the image comparison scheme being compared against a next digital image, until a sharpest digital image has been identified from among the plurality of digital images.

19. The non-transitory medium of claim 17, wherein said implementing is performed iteratively, with the sharper digital image identified in each instance of the image comparison scheme being compared against a next digital image, until the first and second metrics converge within a threshold amount.

20. The non-transitory medium of claim 17, wherein said implementing is performed iteratively, with the sharper digital image identified in each instance of the scheme being compared against a next digital image, until a predetermined amount of time elapses, and wherein following a conclusion of the predetermined amount of time, whichever digital image is presently identified as sharpest is identified as the sharpest digital image.
